(19) United States
(12) Patent Application Publication
(10) Pub. No.: US 2010/0086994 A1
(43) Pub. Date: Apr. 8, 2010

(54) Structure of fermentation apparatus

(75) Inventors: Yung-Hsiang Lin, Sijhih City (TW); Hung-Lin Chen, Banciao City (TW)

Correspondence Address: ROGER H. CHU, 19499 ERIC DRIVE, SARATOGA, CA, 95070, US

(73) Assignee: TCI CO., LTD., Taipei City (TW)

(21) Appl. No.: 12/246,623

(22) Filed: Oct. 7, 2008

(51) Int. Cl. C12M 1/06

(52) U.S. Cl. 435/289.1

(57) ABSTRACT

A fermentation apparatus includes a fermentation tank, an air-hole mesh board, a lift-up cover, a blender and a pressurization equipment. An opening is above fermentation tank, an oblique-board base is at the bottom of the fermentation tank, and a feed opening and an air outlet are on the fermentation tank. Two air inlet holes are on the oblique-board base to form a passage through the oblique-board base. Exhaustion holes are on a surface of the oblique-board base and interconnected with the passage. The air-hole mesh board is installed in the fermentation tank and between the feed opening and the oblique-board base. The lift-up cover is connected to the fermentation tank and disposed above the fermentation tank for covering the opening. The blender is between the lift-up cover and the air-hole mesh board. The pressurization equipment is connected to the air inlet hole for supplying a high-pressure gas into fermentation tank.

### FIELD OF THE INVENTION

The present invention generally relates to a fermentation apparatus, and more particularly to an improved structure of a fermentation apparatus installed in a fermentation tank having an oblique-board base structure.

### BACKGROUND OF THE INVENTION

Fermentation apparatus is mainly used for converting substrates (or nutrients) into products by microorganisms in a fermentation tank. In a conventional fermentation tank, a blending equipment is provided for connecting and mixing a gas for a chemical reaction to achieve the aforementioned fermentation process of converting substrates into products, and some fermentation tanks used for researches are connected to a number of monitoring devices or measuring devices, and thus the fermentation apparatus usually comes with a high price.

In recent years, we pay more attentions on environmental protection and health, and thus there is an increasingly higher demand for different products including fermented diary products, red yeast rice related food, beers, and yeast related products produced by the fermentation process, and these products have become extensively used, and thus it is an important subject for the related industry to improve the production capacity of the fermented products.

In view of the foregoing need, the inventor of the present invention provides an improved structure of a fermentation apparatus, particularly a fermentation apparatus of a fermentation tank having an oblique-board base structure, wherein the oblique-board base structure is used for increasing the flow rate of a high-pressure gas and scattering the high-pressure gas quickly, so that the gas can be contacted with the surface of a desired fermenting substance effectively and stably to enhance the efficiency and speed of a fermentation process, while maintaining a high quality of the fermented substance and achieving the mass production of the fermented products.

### BRIEF SUMMARY OF THE INVENTION

Therefore, it is a primary objective of the present invention to provide an improved structure of a fermentation apparatus, particularly a fermentation apparatus of a fermentation tank having an oblique-board base structure.

The improved structure of the fermentation apparatus of the invention comprises a fermentation tank, an air-hole mesh board, a lift-up cover, a blender and a pressurization equipment, and the fermentation tank has an opening formed at the top of the fermentation tank, a feed opening and an air outlet formed respectively at appropriate positions on an upper half of a sidewall of the fermentation tank, and an oblique-board base disposed at the bottom of the fermentation tank. The sidewall of the oblique-board base has two air inlet holes for forming a passage through the interior of the oblique-board base. A surface of the oblique-board base has a plurality of exhaustion holes passing through the interior of the oblique-board base and coupled with the air inlet hole.

The air-hole mesh board is installed in a containing space inside the fermentation tank and disposed between the feed opening and the oblique-board base, and the feed opening and the air outlet are disposed above the air-hole mesh board. The lift-up cover is a transparent plastic board made of a PC material, and the lift-up cover is connected to the fermentation tank and disposed above the opening of the fermentation tank for covering the opening. The blender is disposed in the fermentation tank and between the lift-up cover and the air-hole mesh board. The pressurization equipment is coupled to the two air inlet holes of the fermentation tank for supplying a high-pressure gas into the fermentation tank.

To make it easier for our examiner to understand the technical characteristics and effect of the present invention, we use preferred embodiments with accompanying drawings for the detailed description of the invention. However, the embodiments and drawings are provided for illustrating the invention, but not intended for limiting the scope of the invention.

### BRIEF DESCRIPTION OF THE DRAWINGS

FIG. 1 is a perspective view of a fermentation tank preferred embodiment of the present invention;

FIG. 2 is a side view of a fermentation tank preferred embodiment of the present invention; and

FIG. 3 is a top view of a fermentation tank preferred embodiment of the present invention.

### DETAILED DESCRIPTION

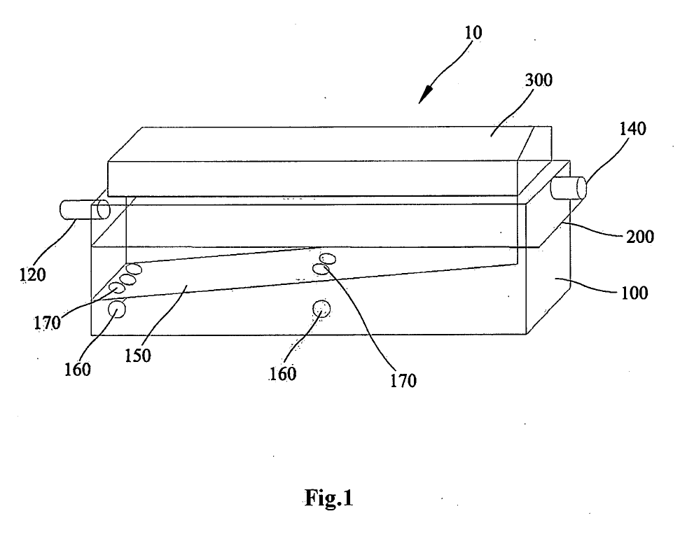

With reference to FIG. 1 for an improved structure of a fermentation apparatus in accordance with a preferred embodiment of the present invention, the fermentation apparatus 10 in a fermentation tank 100 is a box structure, and the fermentation tank 100 has an opening (not shown in the figure) formed at the top of the fermentation tank 100, and a feed opening 120 and an air outlet 140 corresponding to each other and disposed respectively at appropriate positions on an upper half of a sidewall of the fermentation tank 100, and an oblique-board base 150 disposed at the bottom of the fermentation tank 100. The sidewall of the oblique-board base 150 has two air inlet holes 160 penetrated deep into the oblique-board base 150 to form a passage passing through the interior of the oblique-board base 150. The plurality of exhaustion holes 170 are disposed on a surface of the oblique-board base 150 and passed through the interior of the oblique-board base 150, and perpendicularly penetrated into the passage formed by the air inlet hole 160, and coupled with the air inlet hole 160.

An air-hole mesh board 200 is installed in a containing space inside the fermentation tank 100 and disposed between the feed opening 120 and the oblique-board base 150. A lift-up cover 300 is coupled with the fermentation tank 100 and disposed above the opening of the fermentation tank 100 for covering the opening, and the lift-up cover 300 is a transparent plastic board made of a PC material and provided for users to observe the fermentation process of a desired fermenting substance in the fermentation tank 100. A blender (not shown in the figure) is installed between the lift-up cover 300 and the air-hole mesh board 200 in the fermentation tank for blending the desired fermenting substance and the high-pressure gas in the fermentation tank 100 to expedite the fermentation process.

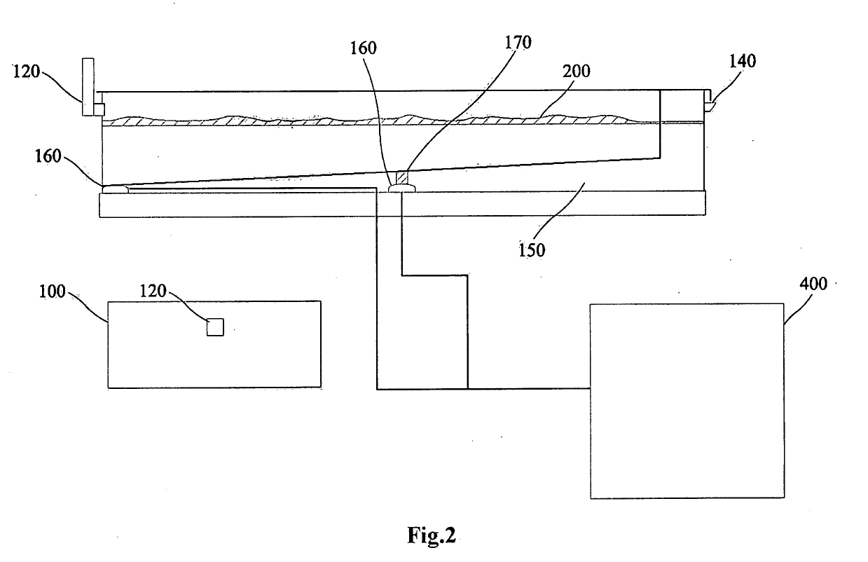

With reference to FIG. 2 for a side view of an improved structure of a fermentation apparatus in accordance with a preferred embodiment of the present invention, a user stands next to the feed opening 120 at the top of the air-hole mesh board 200 pours a desired fermenting substance onto the air-hole mesh board 200 in the fermentation tank 100, and an air compressor 400 is connected to the air inlet hole 160 for supplying a high-pressure gas into the fermentation tank 100 and filling the high-pressure gas from the exhaustion holes 170 into the fermentation tank 100, and a blender starts blending the desired fermenting substance and the high-pressure gas. The transparent lift-up cover 300 is provided for users to observe the fermentation process clearly and control the fermentation condition of the desired fermenting substance. In the meantime, the air outlet 140 above the air-hole mesh board 200 is provided for discharging waste gases produced in the fermentation process to the outside through the air outlet 140.

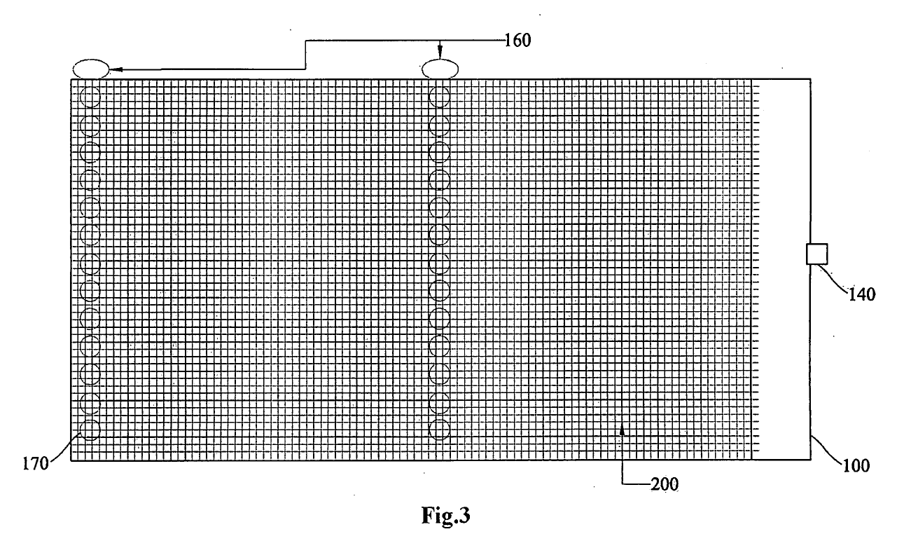

With reference to FIG. 3 for a top view of a fermentation tank of an improved structure of a fermentation apparatus in accordance with a preferred embodiment of the present invention, when the high-pressure gas is guided through the air inlet hole 160 and passed from the air compressor 400 (refer to FIG. 2 together) into the fermentation tank 100, the high-pressure gas is also guided through the exhaustion hole 170 of the oblique-board base 150 into the fermentation tank 100. The inclination of the oblique-board base 150 preferably falls within the range of 0.2 and 0.033, and the diameter of the exhaustion hole 170 is 0.5 mm to achieve better flow and efficiency of the high-pressure gas. In addition, the speed of inputting the high-pressure gas by the operation of the air compressor depends on the speed of the gas flow to the fermentation tank 100 during the fermentation process.

By taking the mobility of the desired fermenting substance in a gaseous state, the volume of the desired fermenting substance in a solid state, and the size of air bubbles of the high-pressure gas into consideration, we can set the diameter of the holes on the air-hole mesh board 200 to 0.2 mm and 16 mesh to achieve a better effect.

In summation of the description above, the present invention improves over the prior art and complies with the patent application requirements, and thus is duly filed for patent application. While the invention has been described by means of specific embodiments, numerous modifications and variations could be made by those skilled in the art without departing from the scope and spirit of the invention set forth in the claims.

What is claimed is:

1. An improved structure of a fermentation apparatus, comprising:
a fermentation tank, having an opening formed on the top of the fermentation tank, a feed opening and an air outlet disposed at appropriate positions on an upper half of a sidewall of the fermentation tank respectively, and an oblique-board base disposed at the bottom of the fermentation tank, and the fermentation tank comprising:
at least two air inlet holes, deposed on a sidewall of the oblique-board base, and forming a passage passing through the interior of the oblique-board base; and
a plurality of exhaustion holes, disposed on an upper surface of the oblique-board base, and passed through the interior of the oblique-board base, and perpendicularly penetrated to the air inlet hole to form the passage, and coupled with the air inlet hole;
an air-hole mesh board, installed in a containing space inside the fermentation tank, and disposed between the feed opening and the oblique-board base;
a lift-up cover, coupled to the fermentation tank, and disposed above the opening of the fermentation tank, for covering the opening;
a blender, disposed in the fermentation tank, and installed between the lift-up cover and the air-hole mesh board; and
a pressurization equipment, coupled to the two air inlet holes of the fermentation tank, for supplying a high-pressure gas into the fermentation tank.

2. The improved structure of a fermentation apparatus of claim 1, wherein the oblique-board base of the fermentation tank has an inclination falling within a range between 0.2 and 0.033.

3. The improved structure of a fermentation apparatus of claim 1, wherein the feed opening and the air outlet of the fermentation tank are disposed above the air-hole mesh board.

4. The improved structure of a fermentation apparatus of claim 1, wherein the plurality of exhaustion holes of the oblique-board base have a hole diameter of 0.5 mm.

5. The improved structure of a fermentation apparatus of claim 1, wherein the air-hole mesh board has air holes with a diameter selectively set to 0.2 mm and 16 mesh.

6. The improved structure of a fermentation apparatus of claim 1, wherein the lift-up cover is a transparent plastic board made of a PC material.

7. The improved structure of a fermentation apparatus of claim 1, wherein the pressurization equipment is an air compressor.
